(19) United States
(12) Patent Application Publication
(10) Pub. No.: US 2008/0032917 A1
(43) Pub. Date: Feb. 7, 2008

(54) FLAVIVIRUS PROTEASE SUBSTRATES AND INHIBITORS

(75) Inventors: Jun Li, San Diego, CA (US); Jennifer Leslie Harris, San Diego, CA (US); Christine Tumanut, Spring Valley, CA (US)

Correspondence Address: GENOMICS INSTITUTE OF THE;NOVARTIS RESEARCH FOUNDATION, 10675 JOHN JAY HOPKINS DRIVE, SUITE E225, SAN DIEGO, CA, 92121-1127, US

(73) Assignee: IRM LLC, A DELAWARE LIMITED LIABILITY COMPANY, Hamilton (BM)

(21) Appl. No.: 11/619,155

(22) Filed: Jan. 2, 2007

Related U.S. Application Data
(63) Continuation-in-part of application No. PCT/US05/21995, filed on Jun. 23, 2005.
(60) Provisional application No. 60/585,797, filed on Jul. 3, 2004.

(51) Int. Cl. A61K 38/00; C07K 2/00; C07K 5/00; C07K 7/00; C12N 5/00

(52) U.S. Cl. 514/2; 435/375; 530/300; 530/328; 530/330

(57) ABSTRACT

The invention provides substrate specificity profiles for flaviviral proteases (e.g., dengue proteases or West Nile protease). Optimal flaviviral protease substrate sequences, both to the prime side and non-prime side of the flaviviral protease recognition site, are disclosed herein. The flaviviral protease substrate sequences are used in designing substrates, inhibitors, and prodrugs. Flaviviral protease inhibitors based on substrate specificity are also provided.

### CROSS-REFERENCE TO RELATED APPLICATIONS

The subject patent application is a continuation-in-part of and claims the benefit of priority under 35 U.S.C. 365(c) to international application No. PCT/US05/21995 (filed Jun. 23, 2005). The aforementioned international application in turn claims the benefit of priority to U.S. Provisional Patent Application No. 60/585,797 (filed Jul. 3, 2004). The full disclosures of these previously filed applications are incorporated herein by reference in their entirety and for all purposes.

### FIELD OF THE INVENTION

The present invention relates to substrate specificity of dengue proteases and substrate design. More particularly, the present invention relates to inhibitors for targeting dengue protease enzyme activity.

### BACKGROUND OF THE INVENTION

Substrate specificity of an enzyme is an important characteristic that governs its biological activity. Characterization of substrate specificity provides invaluable information useful for a complete understanding of often complex biological pathways. In addition, substrate specificity profiles are useful in the design of selective substrates, inhibitors, and prodrugs directed to enzymatic targets. Proteases, also known as proteinases, peptidases, or proteolytic enzymes, are enzymes that degrade proteins by hydrolyzing peptide bonds between amino acid residues. Various categories of proteases include thiol proteases, acid proteases, serine proteases, metalloproteases, cysteine proteases, carboxyl proteases, and the like.

Infection of flaviviruses (e.g., dengue virus or West Nile virus) is the cause of several serious infectious diseases in human subjects. Examples include dengue fever, West Nile encephalitis, Japanese encephalitis and yellow fever. For example, dengue fever is an endemic viral disease with around 50 million infection cases reported worldwide every year. There are four antigenically distinct serotypes of dengue virus, Den 1-4, with Den-2 being the most prevalent in many recent epidemics. There has been no effective treatment for any of the four dengue serotypes. Dengue viruses use their ˜11 kb ss (+) RNA genome as direct template for the synthesis of a single precursor polyprotein. Both host signal peptidases and viral NS3 serine protease are involved in processing the polypeptide into at least 10 viral proteins: the three structural proteins C, prM and E that form the virion particle, and the seven non-structural proteins, NS1 to NS5, that function in the virus life cycle. The NS3 proteases are essential for replication of dengue virus as well as other members of the flaviviridae family (e.g., West Nile virus). Mutations in the NS3 proteases which eliminated proteolytic processing abolished recovery of infectious virus following RNA transfection.

Since NS3 protease has been found essential for viral replication in the Flaviviridae family, there are needs in the art for inhibitors of NS3 proteases of the viruses that are useful in antiviral therapy (e.g., for dengue or West Nile viral infections). By providing selective substrates and novel inhibitors for flaviviral NS3 proteases using tetrapeptide and octapeptide substrate libraries, the present invention fulfills such needs, as well as other needs that will be apparent upon complete review of this disclosure.

### SUMMARY OF THE INVENTION

The present invention provides flaviviral (e.g., dengue or West Nile virus) protease substrates, prodrugs, diagnostics and inhibitors, as well as screening and therapeutic methods involving flaviviral NS3 protease. In one aspect, the invention provides flaviviral NS3 protease-cleavable molecules having a NS3 protease cleavage site. The NS3 protease-cleavable molecules typically comprise P₄P₃P₂P₁X, wherein P₁ is arginine or lysine; P₂ is arginine, lysine, threonine, glutamine, asparagines, leucine, or isoleucine; P₃ is lysine, glycine, arginine, histidine, or asparagine; P₄ is norleucine, leucine, lysine, arginine, or glutamine; and one or more amino acids attached to either or both of P₁ and P₄; and X comprises one or more of an inhibitory moiety, a label moiety, a polypeptide comprising 1 to 25 amino acids, or a polypeptide that is not attached to P₄P₃P₂P₁ in a naturally occurring protein; and wherein the NS3 protease cleavage site is between P₁ and X.

In some embodiments, the P₄P₃P₂P₁ in the NS3 protease-cleavable molecules has a sequence selected from the group consisting of nKRR, nKKR, LKRR, LKKR, nRRR, nRKR, LRRR, LRKR, nGRR, nGKR, nKRK, nKKK, LKRK, LKKK, nKTR, and LKTR (“n” represents norleucine). In some embodiments of the NS3 protease-cleavable molecules, X comprises P₁′P₂′P₃′P₄′, wherein P₁′ is attached to P₁ and is serine or glycine; P₂′ is glycine, aspartic acid, glutamic acid, or alanine; P₃′ is serine or asparagine; and P₄′ is glycine, asparagine, or alanine. In some embodiments, the label moiety comprises a fluorophore, a coumarin moiety, or a rhodamine moiety.

In another aspect, the invention provides flaviviral NS3 protease-cleavable peptides that comprise fewer than 25 amino acids. The peptides comprise P₄P₃P₂P₁, wherein P₁ is arginine or lysine; P₂ is arginine, lysine, threonine, glutamine, asparagines, leucine, or isoleucine; P₃ is lysine, glycine, arginine, histidine, or asparagine; P₄ is norleucine, leucine, lysine, arginine, or glutamine; and one or more amino acids attached to either or both of P₁ and P₄.

In some of these NS3 protease-cleavable peptides, P₁ is arginine or lysine, P₂ is arginine or lysine, P₃ is lysine, glycine, or arginine, and P₄ is norleucine, leucine, or lysine. Some of the peptides further comprise 1 to 20 amino acids linked to P₄. Some of the NS3 protease-cleavable peptides further comprise 1 to 20 amino acids linked to P₁. Some of the peptides further comprise P₁′P₂′P₃′P₄′, wherein P₁ is attached to P₁ and is serine or glycine; P₂′ is glycine, aspartic acid, glutamic acid, or alanine; P₃′ is serine or asparagine; and P₄′ is glycine, asparagine, or alanine.

In a related aspect, the invention provides flaviviral NS3 protease inhibitors. The inhibitors comprise P₄P₃P₂P₁Z, wherein P₁ comprises arginine or lysine; P₂ comprises arginine, lysine, threonine, glutamine, asparagines, leucine, or isoleucine; P₃ comprises lysine, glycine, arginine, histidine, or asparagine; P₄ comprises norleucine, leucine, lysine, arginine, or glutamine; and Z comprises an inhibitory moiety. The inhibitory moiety can be a transition state analog, a mechanism-based inhibitor, or an electron withdrawing group. Some of the NS3 protease inhibitors comprise an inhibitory moiety selected from the group consisting of a C-terminal aldehyde, a boronate, a phosphonate, an α-ketoamide, a chloro methyl ketone, a sulfonyl chloride, ethyl propenoate, vinyl amide, vinyl sulfone, vinyl sulfonamide. In some of the NS3 protease inhibitors, P₁ is arginine or lysine, P₂ is arginine or lysine, P₃ is lysine, glycine, or arginine, and P₄ is norleucine, leucine, or lysine.

In one aspect, methods for identifying a modulator of a flaviviral NS3 protease (e.g., dengue or West Nile NS3 protease) are provided. The methods involve the steps of (a) contacting a test agent with the NS3 protease in the presence of a NS3 protease substrate of the invention, and (b) detecting an alteration of cleavage of the NS3 protease substrate by the NS3 protease in the presence of the test agent relative to cleavage of the NS3 protease substrate by the NS3 protease in the absence of the test agent; thereby identifying a NS3 protease modulator.

In another aspect, the invention provides methods for reducing a flaviviral NS3 protease activity in a cell. The methods entail contacting the cell with a NS3 protease inhibitor molecule of the invention, thereby reducing the flaviviral NS3 protease activity in the cell.

### BRIEF DESCRIPTION OF THE DRAWINGS

FIG. 1 shows results of positional scanning of a P1×P2-position fixed tetrapeptide libraries for dengue 1 NS3 protease.

FIG. 2 shows the non-prime side P₄-P₁ substrate specificity for dengue 1 protease.

FIG. 3 shows the non-prime side P₄-P₁ substrate specificity for dengue 2 protease.

FIG. 4 shows the non-prime side P₄-P₁ substrate specificity for dengue 3 protease.

FIG. 5 shows the non-prime side P₄-P₁ substrate specificity for dengue 4 protease.

FIG. 6 shows one-position fixed donor-quencher substrate library that is customized for dengue NS3 proteases.

FIG. 7 shows prime-side P₁′-P₄′ specificity of dengue NS3 proteases from the four dengue serotypes.

FIG. 8 shows kinetics of NS3 proteases from the four different dengue serotypes on substrates with optimal and suboptimal P4-P1 sequences.

FIG. 9 shows comparison of monitoring dengue 2 NS2B/NS3 protease activities by positional scanning based substrates versus other reported tools.

FIG. 10 shows comparison of P₄-P₄′ substrate preference with in vivo cleavage sites of dengue 2 NS3 proteases.

FIG. 11 shows preferences at P1-P4 positions of substrates of West Nile NS3 protease.

FIG. 12 shows kinetics of West Nile NS3 protease activity on several individual substrates.

### DETAILED DESCRIPTION

Before describing the present invention in detail, it is to be understood that this invention is not limited to particular devices or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a flavivirus (e.g., dengue or West Nile virus) protease substrate” includes a combination of two or more substrates; reference to “bacteria” includes mixtures of bacteria, and the like.

In some embodiments of the present invention, an active flaviviral (e.g., dengue) protease is optionally expressed in an E. coli, baculovirus, or other available expression system and can be used to generate a substrate specificity profile, e.g., a profile comprising primary and extended specificity on the prime and/or non-prime sides of the cleavage site of a flaviviral (e.g., dengue) protease. For example, positional scanning formats are optionally used with tetrapeptide libraries of putative substrates to provide a substrate profile. Substrates are identified, synthesized and tested for the flaviviral protease cleavage. In addition, the substrate profile is optionally used to develop flaviviral protease inhibitors and prodrugs, e.g., compositions that can be selectively activated (e.g., cleaved and released) where they can inhibit the enzymatic activity of a flaviviral protease, thereby treating flaviviral infections or diseases associated with flaviviruses (dengue fever). Furthermore, the specificity information can optionally be used to identify or confirm physiological substrates and biological pathways in which a flaviviral protease (e.g., dengue NS3 protease) operates.

In one embodiment of the present invention, the substrate specificity information obtained for a flaviviral (e.g., dengue) protease is optionally used to design sequences into small molecule substrates. Fluorescence resonance energy transfer or other fluorescent or chromagenic signals can be used to observe the flaviviral protease activity in vitro, ex vivo, or in vivo. In another embodiment, the sequences are optionally designed into a prodrug format in which the drug is only activated and/or released at sites where a flaviviral protease is expressed.

I. Flaviviral Protease and Protease Polypeptides

A typical enzyme of interest in the present invention is a serine protease from viruses of the flaviviridae family (especially the flavivirus genus), such as dengue protease or West Nile protease. For example, dengue proteases from the four different dengue virus serotypes have all been well known and characterized in the art. See, e.g., Chambers et al., Proc. Nat. Acad. Sci. USA 87: 8898-902, 1990; Chambers et al., J. Virol. 67: 6797-807, 1993, Ryan et al. J. Gen. Virology 79: 947-959, 1998; Murthy et al., J Biol Chem 274: 5573-5580, 1999; Murthy et al., J Mol Biol 301: 759-767, 2000; Brinkworth et al., J Gen Virol 80, 1167-1177, 1999 (Den2), and Leung et al., J Biol Chem 276: 45762-71, 2001 (Den2). The virus-encoded dengue protease comprises the amino-terminal 180 amino acids of NS3 (NS3pro) of the polyprotein. It is responsible for cleavage both in cis and in trans to generate viral proteins that are essential for viral replication and maturation of infectious dengue virions. In addition to its protease activity, the carboxyl-terminal region of NS3 encodes both nucleoside triphosphatase and helicase activities (see, e.g., Li et al., J. Virol. 73: 3108-3116, 1999). Similarly, other flaviviruses (e.g., West Nile virus and yellow fever virus) and their proteases have also been well characterized in the art. See, e.g., Anderson et al., Ann N Y Acad. Sci. 951:328-31, 2001; Nall et al., J Biol Chem. 79:48535-42, 2004; J Biol Chem. 280:2896-903, 2005; Hillyer et al., Histochem Cell Biol. 117:431-40, 2002; Chambers et al., J Gen Virol. 86:1403-13, 2005; Scaramozzino et al., Biochem Biophys Res Commun. 294:16-22, 2002; and Bessaud et al., Virus Res. 120:79-90, 2006.

In the present invention, the term “flaviviral protease” is used to refer to any portion of a flaviviral protease (e.g., dengue protease) which exhibits substantially similar cleavage patterns to an intact flaviviral protease molecule. For example, a dengue protease molecule typically comprises the amino-terminal 180 amino acids of the NS3 region. As applied to a flaviviral protease, the terms “polypeptide” and “protein” are used interchangeably. Flaviviral protease polypeptides of the invention include, but are not limited to, proteins, biotinylated proteins, isolated proteins, and recombinant proteins. In addition, the polypeptides or proteins of the invention optionally include naturally occurring amino acids as well as amino acid analogs and/or mimetics of naturally occurring amino acids, e.g., that function in a manner similar to naturally occurring amino acids. In the present invention, flaviviral protease polypeptides or peptides can also optionally contain amino acids analogs, derivatives, isomers (e.g., L or D forms of the amino acids), and/or conservative substitutions of amino acid residues. A conservative substitution refers to the replacement of one amino acid with a chemically-similar residue, e.g., the substitution of one hydrophobic residue for another. Exemplary substitutions include, but are not limited to, substituting alanine, threonine, and serine for each other, asparagine for glutamine, arginine for lysine, and the like.

II. Substrate Libraries for Profiling Flaviviral Protease Substrate Specificity

Recognition of the primary cleavage sequence of the substrate (substrate specificity) is an important mechanism of protease regulation. For many screening applications, e.g., screens for flaviviral protease activity or flaviviral protease substrate specificity profiles, a library of substrates or putative substrates is desired. A “library” is a collection or group of molecules, e.g., about 350-400 or more molecules, about 1000 or more molecules, about 10,000 or more, and/or about 100,000 or more molecules. Typically, each member of the library comprises a different molecule. As such, the number of members in a given library of the present invention is optionally the number of constitutive components, or substrate moiety options (e.g., 19-20 amino acid options), to the power of how many positions are being varied (e.g., 3 positions in a 1-fixed-position tetrapeptide). For example, a library of tetrapeptide substrates generated using 20 amino acids and keeping the P₁ position fixed as arginine can comprise a maximum collection of (20)₃ or 8,000 different peptide sequences that are potentially cleavable by a flavivirus protease such as dengue protease or West Nile protease.

A library of putative flaviviral protease substrates is a library or collection of molecules that may or may not be cleavable by a flaviviral protease. It can be created using peptide synthesis techniques well known to those of skill in the art, or the techniques described in PCT application WO 03/029823 (“Combinatorial Protease Substrate Libraries”). Such a library is used, e.g., to probe substrate specificity. These libraries are optionally used to provide non-prime side information regarding the enzyme active site with respect to the various member substrates of the library. For example, an optimal non-prime substrate sequence, e.g., the first four amino acids on the non-prime side (e.g., N-terminal side) of the cleavage site can be identified for a flaviviral protease (e.g., dengue protease). This information is optionally used to design more selective and/or potent substrates. For example, different fluorogenic compounds are optionally employed to increase the sensitivity (e.g., detection sensitivity) of these substrates. The substrates identified also can provide valuable diagnostics for the identification of protease activity in complex biological samples, and are valuable in screening efforts to identify protease inhibitors.

Members of the substrate libraries or putative substrate libraries typically comprise from about 1 to about 15 substrate moieties, or from about 4 to about 25 substrate moieties. The term “substrate moiety” refers to a component of the substrate molecule, and as such includes any amino acid or amino acid mimetic, as well as the labels, therapeutic molecules, inhibitory molecules described herein, and other components of interest. In addition, selected components are optionally coupled to or linked to the substrates. Such selected components include, but are not limited to: peptides, proteins, non-peptide moieties, sugars, polysaccharides, polyethylene glycol, small molecules, organic molecules, inorganic moieties, label moieties, therapeutic moieties, and/or the like.

Typically, the substrate moieties and selected components, when used in a substrate or putative substrate, form a flaviviral protease cleavage site or a potential flaviviral protease cleavage side. For example, a dengue protease cleaves between two of the substrate moieties, such as between two amino acids or between an amino acid and a coumarin moiety. In some embodiments, the substrate moieties comprise amino acids which provide prime side and/or non-prime side specificity to a flaviviral protease cleavage site. In other embodiments, labels that allow for detection of a cleavage event are incorporated into the substrates of the invention.

Members of the substrate libraries can further comprise a label moiety. The label moiety can be a molecule with fluorescent properties which alter upon cleavage from the substrate, or a matched donor:acceptor pair of fluorescence resonance energy transfer (FRET) compounds. In one embodiment, a fluorescence donor moiety and a fluorescence acceptor moiety are attached to the putative flaviviral protease substrate library members on opposite sides of the putative flaviviral protease cleavage site, such that monitoring the cleavage of the putative flaviviral protease substrates is performed by detecting a fluorescence resonance energy transfer. Monitoring can include detecting a shift in the excitation and/or emission maxima of the fluorescence acceptor moiety, which shift results from release of the fluorescence acceptor moiety from the putative flaviviral protease substrate by the flaviviral protease activity.

In some embodiments, members of the flaviviral protease substrate libraries or putative substrate libraries have one or more positions in the peptide sequence held constant while the others are varied. These libraries, also known as positional scanning libraries, can be created to probe the prime and/or non-prime specificity of a flaviviral protease. As one example, four 20-well sub-libraries can be optionally created, wherein each of the four sub-libraries has a different fixed amino acid position, e.g., P₁, P₂, P₃, or P₄. For example, in a first sub-library, each of the twenty wells contains a library of substrates wherein P₁ is fixed at one of twenty different amino acids, while the other positions, P₂, P₃, and P₄, are varied. In some embodiments of the present invention, the libraries contain about 6859 different substrates per well (i.e., one fixed position and three variable positions per substrate, and using 19 different amino acids during generation of the library, cysteine having been excluded from the synthesis mixture).

Additional sub-libraries can also be optionally created, e.g., with two fixed positions, e.g., P₁/P₂, P₁/P₃, P₁/P₄, P₂/P₃, P₂/P₄, or P₃/P₄. This produces six sub-libraries of 400 wells each (representing each possible combination of the two fixed elements, and the 20 possible elements in each of the fixed positions), wherein each well contains about 361 different substrate sequences (e.g., using the 19 amino acids in the two variable positions). Therefore, the libraries of the invention typically involve about 2400 wells total and the libraries contain well over 100,000 different substrates, e.g., coumarin based substrates. The preferred amino acid for each position, e.g., in a flaviviral protease substrate, is optionally determined using these positional scanning libraries. Positional scanning libraries and methods of using such libraries to determine optimal substrate sequences are described in more detail in the art, e.g., Rano et al., Chemistry and Biology 4, 149-55, 1997; Backes et al., Nature Biotechnology 18: 187-193, 2000; Harris et al., Proc. Natl. Acad. Sci USA 97: 7754-7759, 2000; and Harris et al., Chem. Biol. 8: 1131-1141, 2001.

A non-prime side positional scanning library is typically constructed using a detectable moiety, e.g., a moiety that is not detectable until after it has been cleaved from the substrate (e.g., the peptide). For example, members of a non-prime side scanning library can comprise P₄P₃P₂P₁X, wherein P₄-P₁ comprise amino acids or amino acid mimetics randomized as described above and X comprises a detectable moiety, such as coumarin.

Optionally, prime side specificity can also be analyzed or probed using putative substrate libraries of the present invention. In a preferred embodiment, a prime side position library, e.g., for determining prime side substrate specificity, is constructed using a donor moiety, an acceptor moiety, and a preselected non-prime substrate sequence. Donor moieties and acceptor moieties in the present invention can comprise fluorescence resonance energy transfer pairs. A typical donor moiety for use in the present invention absorbs light at one wavelength and emits at another wavelength, typically a higher wavelength. The acceptor moiety of the invention typically absorbs at the wavelength of either the absorption or emission wavelength of the donor moiety. For example, the acceptor is used as a quencher for the donor moiety. However, the acceptor typically only quenches the absorption or emission of the donor when the two are in proximity, either in high concentrations or when tethered to each other, e.g., chemically bonded. The donor-acceptor pairs are then used to detect protease cleavage (e.g., dengue protease or West Nile protease cleavage) of the substrates of the libraries in the present invention. For example, when cleavage occurs, the acceptor no longer quenches the signal of the donor.

One or more prime position substrate moiety is typically coupled to an acceptor moiety. The prime substrate moieties typically comprise amino acids or amino acid mimetics which are used to form a flaviviral protease cleavable molecule. In a typical library, about four substrate moieties are coupled to the acceptor, e.g., P₁′, P₂′, P₃′, and P₄′. However, the number of substrate moieties coupled to the acceptor is optionally varied, e.g., from about 1 to about 15, but is more typically, about 2 to about 6, and most typically four. Typically, the substrate moieties are coupled to an acceptor using standard peptide synthesis techniques, e.g., Fmoc synthesis.

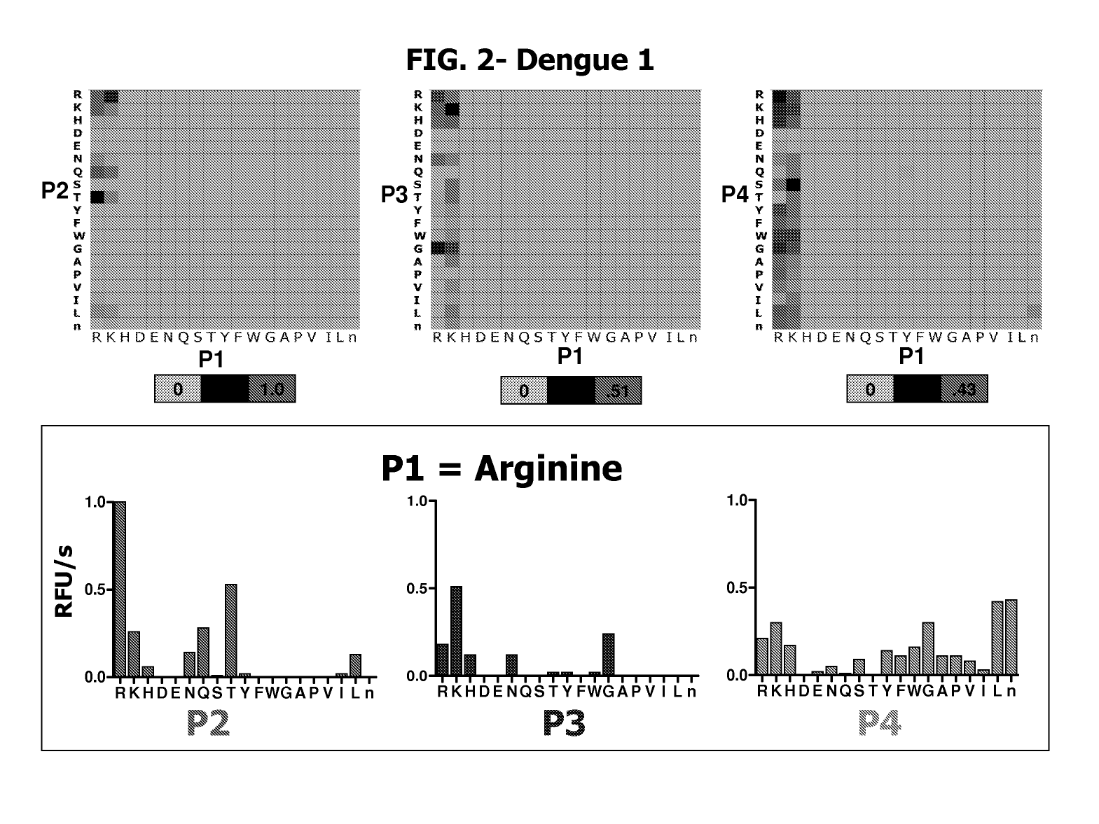

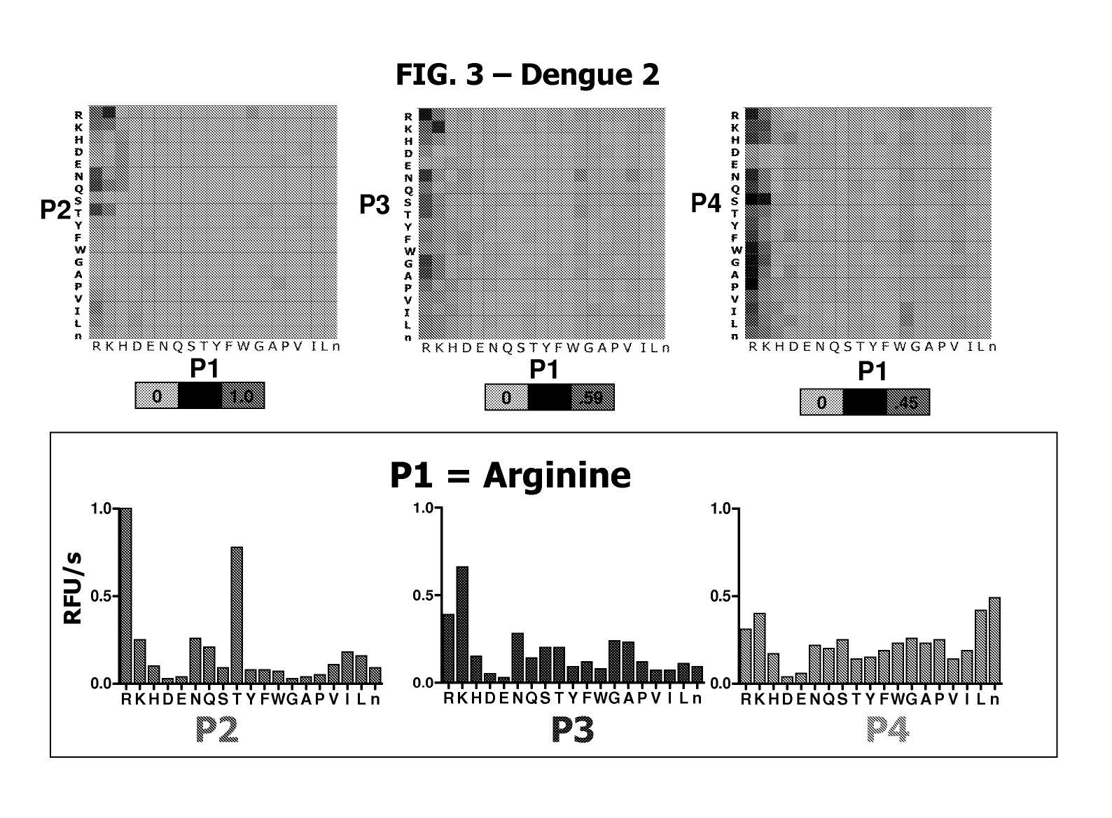

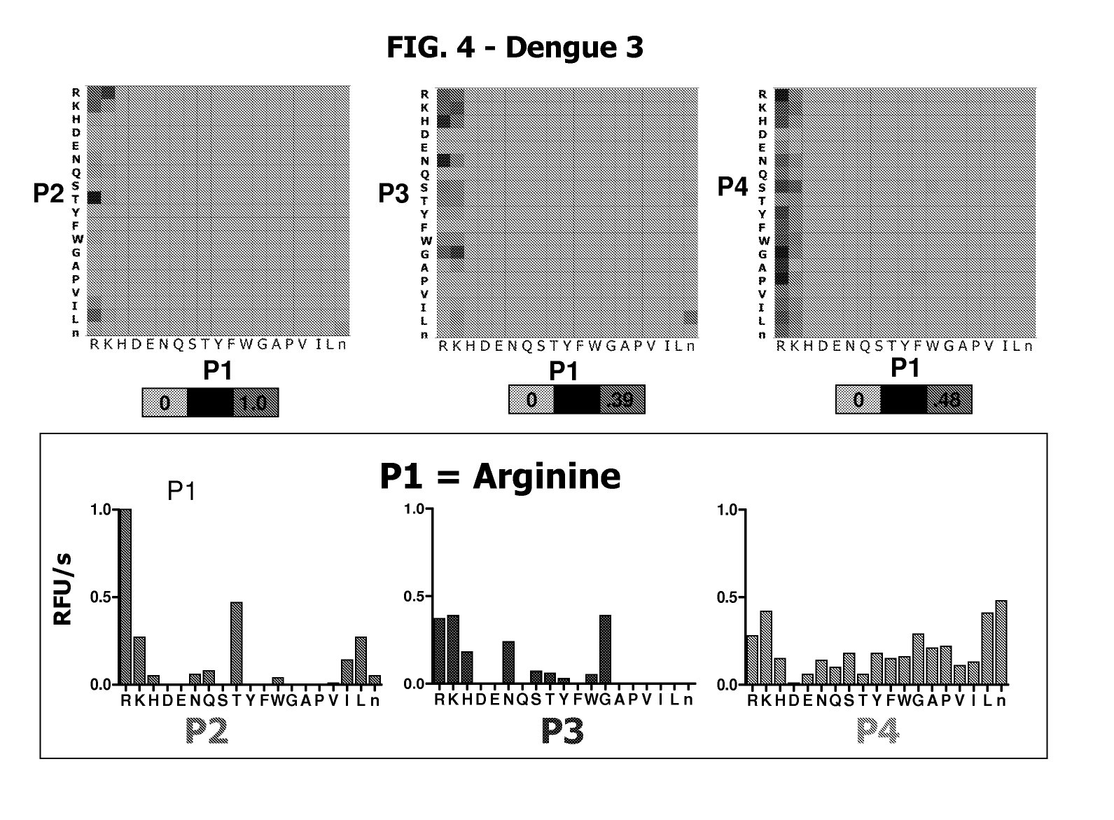

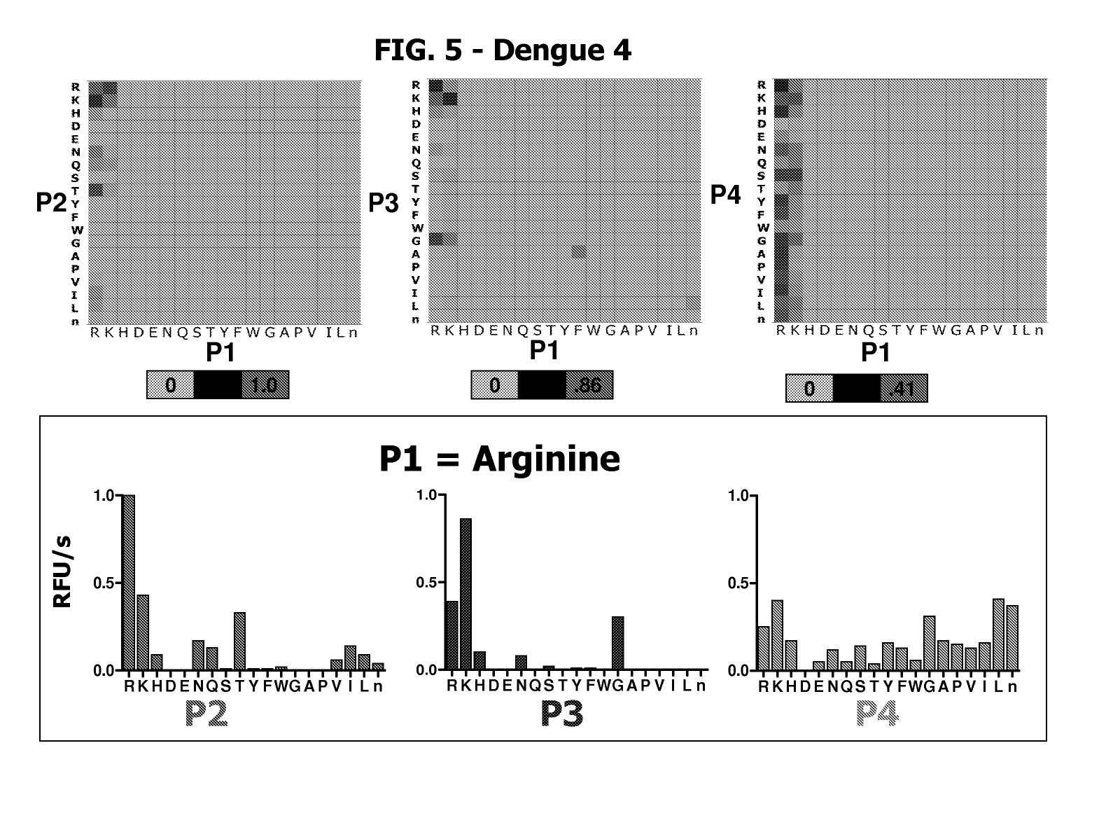

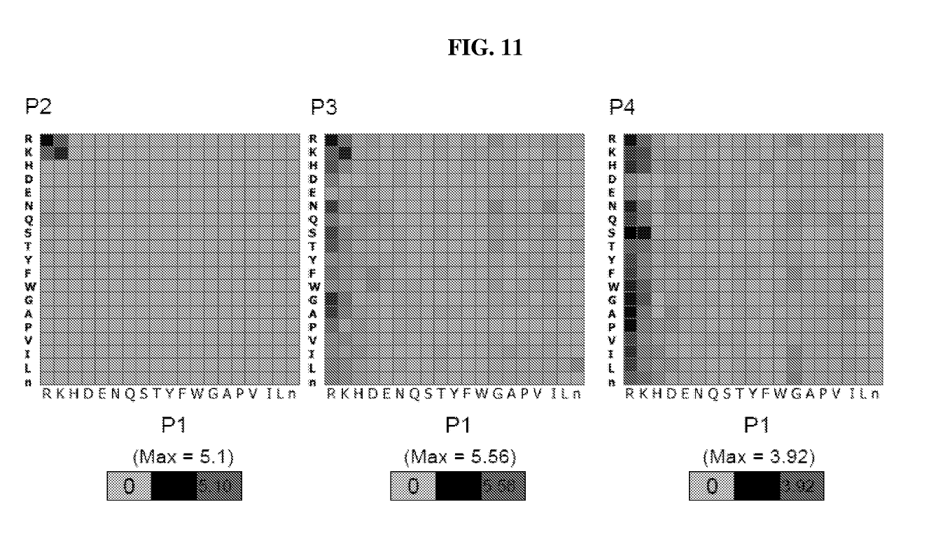

After the prime side positional substrate is coupled to the acceptor, a preselected non-prime substrate, e.g., an optimal or preferred non-prime sequence that has been identified, is coupled to the prime position substrate. “Preselected substrate moieties” are determined as described above and in PCT application WO 03/029823, using, e.g., a positional scanning library. The preselected sequences are typically about 2 to about 20 substrate moieties, e.g., amino acids, in length, more typically about 2 to about 6, and most typically about 4 amino acids or substrate moieties in length. As exemplified in FIGS. 2-5, preselected non-prime side substrate sequences for dengue protease (P₄P₃P₂P₁) could include, e.g., the tetrapeptides nKRR, nKKR, LKRR, LKKR, nRRR, nRKR, LRRR, LRKR, nGRR, nGKR, nKRK, nKKK, LKRK, LKKK, nKTR, and LKTR (“n” represents norleucine). These non-primer side substrate sequences are similarly suitable for other flaviviral proteases, e.g., West Nile protease, as shown in FIG. 11.

III. Profiling Flaviviral Protease Substrate Specificity

The invention provides methods for screening substrate library and profiling substrate specificity of flaviviral proteases (e.g., dengue protease or West Nile protease). To profile a flaviviral protease substrate specificity, a library of flaviviral protease substrates as described above (e.g., a coumarin-based substrate library) is provided. Each member of the library comprises a putative flaviviral protease recognition site. The substrate profile is obtained by monitoring cleavage of the substrates by a flaviviral protease (e.g., dengue protease). Often, to obtain a complete substrate profile for an enzyme, e.g., a protease, a non-prime scan and a prime scan are performed. A “non-prime scan” refers to the scanning library used to determine an optimal substrate sequence for the non-prime side of the cleavage site and/or the results of an analysis of that library. A “prime side scan” refers to the opposite side of the cleavage site, either the library used to probe those positions or the results of such a probe.

Typically, an optimal substrate sequence for the non-prime positions is determined first, using techniques known in the art (e.g., non-prime side scan as exemplified in the Examples below). Thereafter, a second substrate library (e.g., a prime side scan library) is prepared. In some embodiments, a library for a prime scan (e.g., a library for probing prime side substrate sequence specificity) can be prepared using a fluorescence donor-acceptor pair and the optimal non-prime sequences obtained, e.g., as described above. The prime side scan library is then incubated with the enzyme of interest and monitored to determine one or more optimal prime substrate sequence.

As noted above, the substrate moieties that occupy one or more of the non-prime positions can be preselected to allow cleavage of the substrate at the putative flaviviral protease cleavage site by the flaviviral protease (e.g., dengue protease), while allowing the moieties on the prime-side of the cleavage site to vary. Alternatively, both the substrate moieties that occupy the non-prime and the prime positions vary among different members of the library of flaviviral protease substrates (e.g., no pre-selection of library members).

FIGS. 2-5 provide data obtained from incubating a non-prime scan library of coumarin-based substrates with dengue proteases from each of the four different dengue virus serotypes. Similar data obtained for West Nile protease is shown in FIG. 11. The figures depict the enzyme activity for pools of library members having two “fixed” positions in the tetrapeptide-coumarin substrate. When a flaviviral protease acts on a substrate, the substrate is cleaved between P₁ and the coumarin moiety, thereby releasing a fluorogenic coumarin moiety, which is detected. The results indicate that the non-prime side substrate specificities of the four different dengue proteases as well as West Nile protease are substantially identical. As shown in the figures, arginine and lysine are the most preferred P₁ residues. Preferred residues for positions P₂-P₄, based on having a fixed P₁ substituent, are also illustrated in the figures. For example, the preferred residues in the P₂ position are arginine and lysine. The P₃ position prefers lysine, arginine, glycine, histidine, or asparagine. The P₄ position prefers norleucine, leucine, lysine, arginine, or glutamine.

After the non-prime side sequence is determined, a second library can be constructed to determine the prime side substrate specificity of the flaviviral protease (e.g., a dengue protease). The non-prime side sequence of members of this second substrate library sequence is preselected based on the information obtained from the non-prime scan. For example, the non-prime side of the substrate in the second substrate library of the invention can be kept constant as the sequence determined from a coumarin library, P₄-norleucine, P₃-Lys, P₂-Arg, P₁-Arg, or any other sequence as provided above. The prime side four amino acid positions are typically randomized as all 20 natural amino acids. However, in some embodiments, norleucine is optionally used to replace methionine and/or cysteine is optionally excluded.

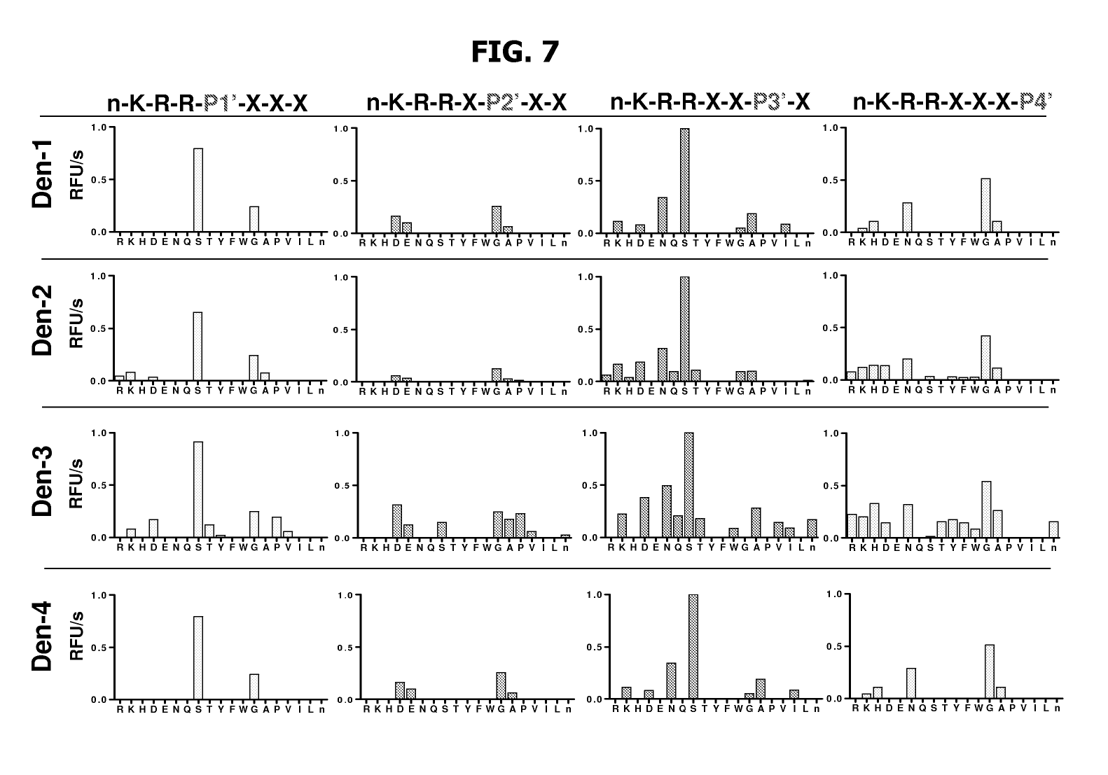

Similar to that of the non-prime side, the prime side substrate specificity of the four dengue proteases are also almost identical. An exemplary primer side specificity profile for each of the four dengue proteases, with the preselected non-prime side sequence being P₄-norleucine, P₃-Lys, P₂-Arg, P₁-Arg, is provided in FIG. 7. The figure shows provide prime side substrate specificity for P₁′, P₂′, P₃′ and P₄′ with the y-axis representing relative fluorescence units per second and the x-axis representing the amino acid held constant in the substrate. The results indicate that the preferred residues for the prime side sequence are P₁′: serine or glycine; P₂′: glycine, aspartic acid, glutamic acid, or alanine; P₃′: serine or asparagine; and P₄′: glycine, asparagine, or alanine.

The prime and non-prime side sequence of a flaviviral protease substrate as determined above can be used to search genomic databases, e.g., for similar cleavage sites in proteins and provide possible macromolecular substrates that are key to the biological function of a flaviviral protease (e.g., dengue protease). In addition, as described in more detail below, the information is useful to design peptide based inhibitors of a flaviviral protease (e.g., a dengue protease or West Nile protease), prodrugs and diagnostic reagents based on the flaviviral protease specificity. The prime and non-prime information can also be used to design more selective and potent substrates, e.g., for use as therapeutic agents or biological tools. Multiple fluorogenic compounds can be employed with the determined amino acid specificity sequence to increase the sensitivity and efficacy of these substrates for a particular system.

Furthermore, substrates of the present invention are valuable as diagnostics for the identification of protease activity in complex biological samples and for screening efforts to identify protease inhibitors. The overall strategy when applied, e.g., to an entire class of proteases, provides panning information that allows for the generation of specific substrates and inhibitors in the context of an entire protease class. The non-prime and prime specificity information can be employed to bias bead-based and phage display methods, to design cleavage sites in fusion proteins or other protein constructs, and to design prodrugs in which the protease target releases an active drug.

IV. Cleavable Substrates of Flaviviral Proteases

In one aspect, the invention provides flaviviral protease-cleavable substrates (e.g., dengue protease cleavable substrate). Typically, such flaviviral protease substrates are peptide-based molecules that are cleavable by a flaviviral protease, including protein, polypeptide and peptide substrates. The substrates also include non-peptide substrates and substrates comprising a peptide attached to a non-peptide moiety. The flaviviral protease recognition sites employed in the present invention typically comprises an amino acid sequence, e.g., about 4 to about 25 amino acids. The amino acids are typically selected to form a flaviviral protease specific cleavage site, e.g., a sequence that is cleavable by the flaviviral protease (e.g., a dengue protease). In addition, the sequence is preferably specific for a flaviviral protease, e.g., it is not cleaved by other non-flaviviral proteases. The recognition site is typically a portion of a flaviviral protease substrate, which is cleaved by a flaviviral protease upon recognition. For example, a recognition site typically comprises one or more residue to which a flaviviral protease binds prior to cleavage. Cleavage yields can range anywhere from about 0.1% to 100% cleavage of the substrate.

In some embodiments of the present invention, the flaviviral protease substrates comprise Pₙ . . . P₄ P₃ P₂ P₁ P₁′ P₂′ P₃′ P₄′ . . . Pₙ′. As used herein, the nomenclature for substrates refers to prime side and non-prime side positions, wherein each Pₙ and Pₙ′ (alternatively referred to as P₋ₙ) is typically a substrate component or moiety, such as an amino acid or amino acid mimetic. Cleavage, e.g., amide bond hydrolysis, typically occurs between P₁ and P₁′ (see, e.g., Schechter and Berger (1968) Biochem. Biophys, Res. Commun. 27:157-62). For example, a flaviviral protease typically cleaves an amide bond between two substrate moieties, such as between an amino acid in a prime side peptide P₁ position and an amino acid in a non-prime side peptide P₁′ position. Optionally, “n” ranges from zero to 21 substrate moieties, thereby providing substrates with various number of units (e.g., amino acids) in length.

In other embodiments, the substrates comprise Pₙ . . . P₄ P₃ P₂ P₁X, wherein X is a selected component such as a peptide, a protein, a label moiety, a therapeutic moiety, or the like. For example, in some embodiments, flaviviral protease cleaves a substrate between P₁ and X, wherein P₁ is a peptide moiety (e.g. an amino acid), and X is a diagnostic moiety such as a coumarin compound which fluoresces upon release from the peptide. In some embodiments, the N-terminal amino acid of the substrate is protected, e.g., by acetylation. Other N-terminal protecting groups such as like Z, Cbz, or succinate can also be employed in the flaviviral protease substrates of the invention.

A peptide or substrate of the invention is “cleavable by” a flaviviral protease (e.g., dengue protease or West Nile protease) if, when mixed with the flaviviral protease molecule, the substrate or peptide is cleaved, e.g., at a cleavage site as described above, e.g., between the P₁ and P₁ positions or between P₁ and X. The flaviviral protease substrates of the invention typically comprises a non-prime side sequence (e.g., to the N-terminal side of the cleavage site) and an additional moiety, e.g., a prime side sequence (e.g., to the C-terminal side of the cleavage site), a therapeutic moiety, or a diagnostic moiety (e.g., a fluorophore). When a substrate molecule is cleaved by a flaviviral protease, the additional moiety is released from the peptide upon cleavage, unless the additional moiety is coupled to the substrate molecule at a second position distal from the cleavage site.

Some of the flaviviral protease-cleavable substrates of the present comprise a tetrapeptide sequences in which P₁ is arginine or lysine, P₂ is arginine or lysine, P₃ is lysine, glycine, or arginine, and P₄ is norleucine, leucine, or lysine. In some embodiments, the amino group of the N-terminal amino acid (e.g., P₄) is derivatized or blocked. In some of the substrates, the N-terminal amino acid of the tetrapeptide is N-acetylated. Preferably P₄ is selected from the group consisting of norleucine, leucine, or lysine. Preferred P₄-P₁ peptides for use in the flaviviral protease-cleavable molecules of the present invention include nKRR, nKKR, LKRR, LKKR, nRRR, nRKR, LRRR, LRKR, nGRR, nGKR, nKRK, nKKK, LKRK, LKKK, nKTR, and LKTR (“n” represents norleucine), as illustrated in FIGS. 2-5 and 11.

In addition to the above described peptide sequences, the flaviviral protease cleavable molecules of the present invention can comprise an additional component X, wherein X comprises a therapeutic moiety, a label moiety, a polypeptide (e.g., comprising from about 1 to about 25 amino acids, such as the a prime-side coupled peptides described herein), or a non-native or non-naturally occurring peptide sequence, e.g., one not found in a naturally-occurring flaviviral protease substrate. Other X components that are optionally included in the flaviviral protease substrates of the invention include, but are not limited to: polyalcohols such as polyethylene glycol, biotin, various carbohydrates or carbohydrate polymers, or crosslinking agents. The X component can be coupled or attached to the flaviviral protease cleavable molecule at either or both of the P₁ and P₄ moieties. In some embodiments, the flaviviral protease cleavable molecules are provided in the format P₄P₃P₂P₁X and are cleavable by a flaviviral protease between the P₁ moiety of the peptide sequence and the X component.

In some embodiments, component X comprises a prime-side peptide or peptide-like sequence (the units of which are designated Pₙ′, or sometimes P₋ₙ). For example, a flaviviral protease (e.g., dengue protease) cleavable molecule or flaviviral protease substrate of the invention optionally comprises a non-prime side sequence and a prime side sequence as described above (e.g. Pₙ . . . P₄P₃P₂P₁P₁′P₂′P₃′P₄′ . . . Pₙ). Preferred prime sequences are those in which P₁′ is serine or glycine; P₂′ is glycine, aspartic acid, glutamic acid, or alanine; P₃′ is serine or asparagine; and P₄′ is glycine, asparagine, or alanine, as illustrated in FIG. 7.

Once a flaviviral protease substrate sequence is determined, e.g., from a positional scanning library as described below or by other methods known in the art, the substrate peptides of the present invention are typically synthesized using any recognized procedure in the art, e.g., solid phase synthesis, e.g., t-boc or fmoc protection methods, which involve stepwise synthesis in which a single amino acids is added in each step starting with the C-terminus. See, e.g., Fmoc Solid Phase Peptide Synthesis: A Practical Approach in the Practical Approach Series, by Chan and White (Eds.), 2000 Oxford University Press. The peptides are then optionally used to provide substrates, inhibitors, prodrugs, or diagnostics as described herein.

In forming the various prodrugs, diagnostics, inhibitors, and the like, the peptide sequences provided herein are optionally linked to non-peptide moieties, e.g., aldehydes, cytotoxic compounds, labels, or other additional components. As detailed below, such non-peptide moieties are typically coupled to the peptide sequences, either directly, e.g., via a covalent bond (such as an amide bond or carbamate linkage), or indirectly via a linker molecule (such as a glycol linker or Rink linkers).

V. Flaviviral Protease Substrate Based Prodrugs

A “prodrug” is a composition that is modified to become active, often in vivo. Such compositions typically comprise a therapeutic moiety or cell modulating moiety that is cleaved from the remainder of the composition, preferably at a target site. The therapeutic or cell-modulating moiety is typically activated only after cleavage from the remainder of the composition. The prodrugs of the invention are typically peptides (e.g., the dengue protease-cleavable peptide substrates described above) linked to therapeutic moieties. The therapeutic moieties can be linked to the flaviviral protease substrate peptides either directly or indirectly, e.g., via a covalent bond, or a spacer or linker molecule. The attachment or linkage of the therapeutic moiety to the peptide moiety of the invention typically results in limiting the function of the moiety while attached to the peptide. The moiety is then activated or available for use after being cleaved from the peptide. Therefore, the prodrugs of the invention are not generally toxic. For example, a therapeutic moiety has an effect only when cleaved, e.g., in the presence of a flaviviral protease.

A “therapeutic moiety” of the invention is a compound, molecule, substituent, or the like, that relates to the treatment or prevention of a disease or disorder, e.g., to provide a cure, assist in a cure or partial cure, or reduce a symptom of the disease or disorder. In the present invention, therapeutic moieties are typically linked to the carboxyl terminus of the peptides of the invention, e.g., at P₁. The therapeutic moiety or drug is optionally linked directly to the peptide or via a linker. Direct linkage typically involves an amide bond or an ester bond. When a linker is used, any type of linkage or bond known to those of skill in the art is optionally used.

When a linker is used to attach the therapeutic moiety to the peptide portion of the prodrug, the linker is optionally cleaved from the peptide moiety along with the therapeutic moiety, or it remains attached. If the linker remains with the therapeutic moiety after cleavage by a flaviviral protease, it does not typically affect the function or toxicity of the therapeutic moiety. In other embodiments, the linker or spacer group is self-cleaving. Self-cleaving or self-immolative linkers are those designed to cleave or spontaneously eliminate from the therapeutic moiety after cleavage of the therapeutic moiety from the peptide. For information on self-cleaving linkers useful in prodrugs, see, for example, U.S. Pat. No. 6,265,540 B1.

When administered to a subject, the prodrugs of the invention are typically provided in an aqueous or non-aqueous solution, suspension, or emulsion. Suitable solvents are known to those of skill in the art and include, but are not limited to, polyethylene glycol, ethyl oleate, water, saline, and the like. Preservatives, and other additives are also optionally included, e.g., antimicrobials.

VI. Flaviviral Protease Substrate Based Diagnostics

In addition to prodrugs, the flaviviral protease-cleavable substrates of the present invention are also used as diagnostic reagents or components thereof. For example, a flaviviral protease-cleavable substrate of the invention is optionally linked to a fluorescent molecule, e.g., one that fluoresces only after cleavage from the substrate, to provide a diagnostic moiety that is used to detect the presence of flaviviral protease (e.g., a dengue protease) or in high throughput screening of flaviviral protease inhibitors.

A “diagnostic moiety” is a compound, molecule, substituent, or the like, that is used, e.g., to distinguish or identify, e.g., a certain disease, condition, or diagnosis. For example, the presence of a flaviviral protease is an example of a condition that a diagnostic of the invention is optionally used to identify. A diagnostic moiety of the invention is typically a label moiety that fluoresces upon cleavage from a flaviviral protease substrate and allows the detection of the cleavage event, e.g., that is used to detect the presence of a flaviviral protease.

A “label moiety” is any detectable compound, molecule, or the like. When attached to a flaviviral protease substrate of the invention, the labels provide for detection of flaviviral protease. Typically, the labels of the present invention do not become detectable until after a cleavage event has occurred, e.g., cleaving the label from a flaviviral protease substrate. A label is detectable by any of a number of means, such as fluorescence, phosphorescence, absorbance, luminescence, chemiluminescence, radioactivity, colorimetry, magnetic resonance, or the like.

Label moieties of the invention include, but are not limited to, absorbent, fluorescent, or luminescent label moieties. Exemplary label moieties include fluorophores, rhodamine moieties, and coumarin moieties (e.g., such as 7-amino-4-carbamoylcoumarin, 7-amino-3-carbamoylmethyl-4-methylcoumarin, or 7-amino-4-methylcoumarin). Typically, a label moiety exhibits significantly less absorbance, fluorescence or luminescence when attached to the flaviviral protease-cleavable molecule than when released from the flaviviral protease-cleavable molecule. For example, a fluorophore emits light when it is exposed to the wavelength of light at which it fluoresces. The emitted light is detected. In the present invention, fluorophores with attenuated fluorescence until separated from the attached peptide are typically used. Therefore, a flaviviral protease-cleavable substrate with an attached fluorophore has attenuated fluorescence or provides a diminished signal until the substrate is cleaved by a flaviviral protease, thereby releasing the fluorophore. In this manner, the presence of a flaviviral protease is easily detected using the substrates of the invention. Fluorophores of interest include, but are not limited to, fluorescein, fluorescein analogs, BODIPY-fluorescein, arginine, rhodamine-B, rhodamine-A, rhodamine derivatives, green fluorescent protein (GFP), and the like. For further information on fluorescent label moieties and fluorescence techniques, see, e.g., Handbook of Fluorescent Probes and Research Chemicals, by Richard P. Haugland, Sixth Edition, Molecular Probes, (1996).

An exemplary label moiety that does not fluoresce until cleaved from the substrate is a coumarin moiety. A “coumarin moiety” is a compound or molecule comprising a coumarin compound. Coumarin compounds of interest in the present invention include, but are not limited to, 7-amino-4-carbamoylmethylcoumarin (“acc”), 7-amino-4-methylcoumarin (“amc”), 7-methoxy-4-carbamoylmethylcoumarin, and 7-dimethylamino-4-carbamoylmethylcoumarin, and the like. Many other coumarin compounds are available, e.g., either commercially (see, e.g., Sigma and Molecular Probes catalogs) or using various synthetic protocols known to those of skill in the art. The synthesis of an exemplary coumarin compound of interest is described in WO 03/029823.

The substrates linked to a coumarin moiety can have the non-prime and/or prime side amino acid sequences as provided above. For basic strategies for preparation of and use of coumarin-based substrates and coumarin libraries, see, e.g., Zimmerman et al. (1977) Analytical Biochemistry 78:47-51; Lee et al. (1999) Bioorganic and Medicinal Chemistry Letters 9:1667-72; Rano et al., supra; Schechter and Berger (1968) Biochemical and Biophysical Chemistry Communications 27:157-162; Backes et al. (2000) Nature Biotechnology 18:187-193; Harris et al. (2000) “Rapid and general profiling of protease specificity by using combinatorial fluorogenic substrate libraries” Proc. Natl. Acad. Sci. USA 97:7754-7759; and Smith et al. (1980) Thrombosis Res. 17:393-402.

In other embodiments, quantum dots are optionally used as diagnostic moieties. Nanocrystals, e.g., semiconductor nanocrystals or quantum dots such as cadmium selenide and cadmium sulfide, are optionally used as fluorescent probes. Quantum dots typically emit light in multiple colors, which allows them to be used to label and detect several compounds or samples at once. See, e.g., Bruchez et al. “Semiconductor Nanocrystals as Fluorescent Biological Labels,” Science 281:2013-2016 (1998). Quantum dot probes are available, e.g., from Quantum Dot Corporation (Hayward, Calif.).

In the present invention, a quantum dot is optionally linked to or associated with a flaviviral protease-cleavable substrate and used to detect the substrate, e.g., after cleavage by a flaviviral protease (e.g., a dengue protease or West Nile protease). In some embodiments, the label moiety optionally comprises a first quantum dot attached to a flaviviral protease cleavable molecule on one side of the flaviviral protease cleavage site and a second quantum dot attached to the molecule on the opposite side of the flaviviral protease cleavage site. Typically, the first and second quantum dots emit signals of different wavelengths upon illumination. For example, a quantum dot is optionally linked to a prime side of a peptide substrate as described above, e.g., using standard chemistry techniques, and a differently colored quantum dot is linked to the non-prime side of the substrate. Detection of the quantum dots allows detection of a cleavage event when the prime and non-prime sides are cleaved from each other, e.g., by a flaviviral protease.

Alternatively, electroactive species, useful for electrochemical detection, or chemiluminescent moieties, useful for chemiluminescent detection, are incorporated into the flaviviral protease-cleavable substrates or putative substrates of the invention. UV absorption is also an optional detection method, for which UV absorbers are optionally used. Phosphorescent, colorimetric, e.g., dyes, and radioactive labels are also optionally attached to the flaviviral protease substrates of the invention, e.g., using techniques well known to those of skill in the art.

Labels as described above are typically linked to the flaviviral protease substrates of the invention using techniques well known to those of skill in the art. For example, the label or diagnostic moiety is typically linked to P₁ as P₄P₃P₂P₁X, wherein X comprises the label moiety. Alternatively, the label moiety is linked to the prime side of a flaviviral protease substrate or to P₄. In some embodiments, the label moiety comprises two labels, such as two quantum dots. One label is attached to the prime side of the substrate and the other label is attached to the non-prime side of the substrate, as ′X₁P₄P₃P₂P₁P₁′P₂′P₃′P₄′X₁′, wherein X₁ and X₁′ each comprise a label moiety, such as quantum dot or a member of a FRET pair. In other embodiments, the label moiety is optionally attached to any of the substrate moieties, e.g., P₄-P₁, or P₁′-P₄′. P₄-P₁ and P₁′-P₄′ can be the amino acid sequences as described above for the non-prime and prime sides, respectively, of flaviviral protease recognition site.

The present invention also provides methods of labeling a cell using the labeled flaviviral protease-cleavable molecules of the present invention. The labeling method include contacting the cell with a flaviviral protease-cleavable molecule that comprises a flaviviral protease cleavage site, wherein the dengue protease-cleavable molecule comprises the structure P₄P₃P₂P₁X, wherein the flaviviral protease cleavage site is between P₁ and X; and wherein P₁ is arginine or lysine, P₂ is arginine or lysine, P₃ is lysine, glycine, or arginine, and P₄ is norleucine, leucine, or lysine; and X comprises a label moiety. A variety of labels can be incorporated into the flaviviral protease-cleavable molecules of the present invention, including, but not limited to, a coumarin moiety and members of a donor-acceptor FRET pair, as described herein.

In a further aspect, the present invention provides methods of screening a subject for a flaviviral protease activity or expression, or an increased activity or expression of a flaviviral protease (e.g., a dengue protease or West Nile protease). First, a cell or tissue sample is obtained from the individual. The cell or tissue sample is then put into contact with one or more flaviviral protease-cleavable molecules that comprise a flaviviral protease cleavage site and a detectable label moiety. The flaviviral protease-cleavable molecules can comprise P₄P₃P₂P₁X, wherein the flaviviral protease cleavage site is between P₁ and X; and wherein P₁ is arginine or lysine, P₂ is arginine or lysine, P₃ is lysine, glycine, or arginine, and P₄ is norleucine, leucine, or lysine; and wherein X comprises a label moiety. Any flaviviral protease activity in the sample can be monitored by detecting a signal of the label moiety from the flaviviral protease cleavable molecule. The level of detected label is compared to a control or standard level of flaviviral protease activity, thereby determining whether there is flaviviral protease activity or expression in the subject or an increased activity or expression of a flaviviral protease.

VII. Flaviviral Protease Substrate Based Inhibitors

The invention also provides flaviviral protease inhibitors. Enzyme inhibitors are typically compounds or molecules that negatively affect the ability of an enzyme to catalyze a reaction. A “flaviviral protease inhibitor” is a protease inhibitor that inhibits, curbs, or decreases the activity of a flaviviral protease (e.g., dengue protease or West Nile protease). A typical flaviviral protease inhibitor of the present invention, P₄P₃P₂P₁Z, comprises a flaviviral protease recognition site such as a peptide sequence P₄P₃P₂P₁ as described above. The peptide sequence is typically linked to an inhibitory moiety, Z. An “inhibitory moiety” is a compound or chemical group that is capable of inhibiting a flaviviral protease activity when associated with the flaviviral protease, such as a transition state analog, a mechanism-based inhibitor, an electron withdrawing group, a chemical modifier, or the like. Exemplary inhibitory moieties include, but are not limited to, a C-terminal aldehyde, a boronate, a phosphonate, an α-ketoamide, a chloro methyl ketone, a sulfonyl chloride, ethyl propenoate, vinyl amide, vinyl sulfone, or vinyl sulfonamide.

Mechanism-based inhibitors for various catalytic reactions are well known, e.g., synthetase, peptidase, oxidation/reduction, β-lactamase, decarboxylation, aminotransferase, lyase, racemase, and hydroxylase reactions (Silverman, Chemistry and Enzymology, Vols. I and II, CRC Press, 1988, Boca Raton, Fla.). One of skill in the art can similarly design mechanism based inhibitors for a flaviviral protease based on the prior art disclosure (see, e.g., Silverman, Chemistry and Enzymology, Vols. I and II, CRC Press, 1988, Boca Raton, Fla.; and U.S. Pat. No. 6,177,270). Electron withdrawing groups are also well known in the art. For example, they include chemical groups such as halogen (fluoro, bromo, chloro, or iodo), nitro, trifluoromethyl, cyano, CO-alkyl, CO₂-alkyl, or CO₂-aryl. Chemical compounds such as carboxylic acids, carboxylic acid esters, nitrites, aromatic rings and ketones are all useful electron withdrawing groups.

Transition state analogs for a flaviviral protease can be easily designed and produced. Serine proteases typically have a similar active site geometry, such that hydrolysis of the substrate bond proceeds via the same mechanism of action. The first step in the reaction is the formation of an acyl-enzyme intermediate between the substrate and a conserved serine residue in the active site (hence the classification as a “serine protease”). The peptide bond is cleaved during formation of this covalent intermediate, which proceeds via a (negatively charged) tetrahedral transition state intermediate. Deacylation occurs during the second step of the mechanism of action, at which point the acyl-enzyme intermediate is hydrolyzed by a water molecule, the remaining portion of the substrate peptide is released, and the hydroxyl group of the serine residue is restored. The deacylation process also involves the formation of a tetrahedral transition state intermediate. As such, transition state analog compounds that mimic the structure of either of the tetrahedral intermediates can be employed as inhibitors of the serine protease.

Furthermore, chemical constituents that covalently modify or otherwise interact with the active site of a flaviviral protease molecule (e.g., dengue protease) can also be used as inhibitor moieties in the present invention. In some embodiments of the present invention, cleavage of the flaviviral protease inhibitor molecule irreversibly deactivates the flaviviral protease (e.g., a suicide inhibitor). In other embodiment, the inhibitor moiety need not be released from the flaviviral protease inhibitor molecule to function as an inhibitor (e.g., an inhibitory affinity label). Optionally, the inhibitor moiety is activated upon release from the flaviviral protease recognition site, and functions to either inhibit a single flaviviral protease molecule or to catalyze the inhibition of a number of flaviviral protease molecules. Mechanisms of serine protease inhibition are further described in Fersht (1985) Enzyme Structure and Mechanism (W.H. Freeman and Company, New York).

Typically, the peptide sequence in the flaviviral protease inhibitors is typically based on the substrate specificity of a flaviviral protease (e.g., dengue protease or West Nile protease). For example, the flaviviral protease inhibitors can comprise a P₄-P₁ peptide sequence based on one or more of the flaviviral protease substrates identified above, e.g., nKRR, nKKR, LKRR, LKKR, nRRR, nRKR, LRRR, LRKR, nGRR, nGKR, nKRK, nKKK, LKRK, and LKKK (“n” represents norleucine). Some of the flaviviral protease inhibitors can also comprise an acyl group at their P₄ residue. The transition state analog, mechanism-based moiety, or electron withdrawing moiety in the inhibitors can also comprise a C-terminal aldehyde, a boronate, a phosphonate, an α-ketoamide, a chloro methyl ketone, a sulfonyl chloride, ethyl propenoate, vinyl amide, vinyl sulfone, vinyl sulfonamide, or the like. Thus, an exemplary flaviviral protease inhibitor can comprise Acetyl-P₄-P₃-P₂-P₁-aldehylde, wherein P₄-P₁ comprises a non-prime substrate sequence as provided above.

The aldehyde inhibitor can be prepared using semicarbazone methodology. See, e.g., Dagino and Webb (1994) Tetrahedron Letters 35: 2125-2128. Dagino and Webb describe a method of making peptide aldehydes which involves using a diphenylmethyl semicarbazone group to provide a synthetic intermediate. For example, a protected diphenylmethyl semicarbazide derivative is synthesized, e.g., using techniques known to those of skill in the art. The semicarbazide is reacted to provide a protected argininal derivative, which is converted to a free amine, to which a desired peptide is linked, e.g., using standard peptide coupling techniques. The fully protected peptide aldehydes produced in this manner are optionally purified, e.g., using silica chromatography, and deprotected, e.g., by hydrogenation in acidic aqueous methanol.

VIII. Therapeutic Applications

There are annually ˜50-100 million reported cases of dengue around the world with a mortality rate of ˜25-50 thousand (mostly children). Over 2.5 billion people are at risk of dengue infection. There is no treatment or vaccine that is available today to combat this emerging and uncontrolled disease. Following primary infection, lifelong immunity develops, preventing repeated assault by the same serotype. However, the non-neutralizing antibodies from a previous infection or maternally acquired antibodies are thought to complex with a different serotype from a subsequent infection and cause dengue hemorrhagic fever/dengue shock syndrome, which can be fatal. Similarly, infections by other flaviviruses (e.g., West Nile virus or yellow fever virus) are also associated with severe human diseases.

The flaviviral NS3 protease modulators (e.g., inhibitors) and prodrugs of the present invention can have various applications in treating or preventing diseases caused by the viruses. For example, they are useful in the treatment of West Nile encephalitis, yellow fever, Japanese encephalitis, dengue fever, dengue hemorrhagic fever, and dengue shock syndrome associated with the different dengue virus serotypes. See, e.g., Jacobs et al., Curr. Opin. Infect. Dis. 11: 319-324, 1998; Diamond, Expert Rev Anti Infect Ther. 3:931-44, 2005; Halstead et al., Adv Virus Res. 61:103-38, 2003; and Weir et al., CMAJ. 170:1909-10, 2004. The flaviviral protease inhibitors or prodrugs of the present invention can be used alone or in combination with any known antiviral drugs to treat these infections.

Accordingly, the present invention also provides methods of inhibiting or reducing a flaviviral protease activity in a cell (typically in a subject, e.g., a human subject infected by a dengue virus). The methods involve contacting the cell with a flaviviral protease inhibitor molecule containing a flaviviral protease recognition site. Typically, the flaviviral protease inhibitor molecule comprises a compound comprising the structure P₄P₃P₂P₁X, wherein P₁ is arginine or lysine; P₂ is arginine, lysine, threonine, glutamine, asparagines, leucine, or isoleucine; P₃ is lysine, glycine, arginine, histidine, or asparagine; P₄ is norleucine, leucine, lysine, arginine, or glutamine; and wherein X comprises an inhibitory moiety, such as a transition state analog, a mechanism-based inhibitor, or an electron withdrawing group. Exemplary inhibitory moieties include, but are not limited to, a C-terminal aldehyde, a boronate, a phosphonate, an α-ketoamide, a chloro methyl ketone, a sulfonyl chloride, ethyl propenoate, vinyl amide, vinyl sulfone, or vinyl sulfonamide. In some preferred embodiments, the P₄P₃P₂P₁ portion of the inhibitor comprises a sequence of nKRR, nKKR, LKRR, LKKR, nRRR, nRKR, LRRR, LRKR, nGRR, nGKR, nKRK, nKKK, LKRK, and LKKK (“n” represents norleucine).

To therapeutically or prophylactically treat a disease or disorder, one or more flaviviral protease substrates, inhibitors or prodrugs of the present invention is administered to a subject. Typically, the flaviviral protease inhibitors or prodrugs are administered in pharmaceutical compositions comprising a pharmaceutically acceptable excipient and one or more such flaviviral protease substrates, inhibitors or prodrugs. In these in vivo methods, one or more cells of the subject, or a population of cells of interest, are contacted directly or indirectly with an amount of a flaviviral protease substrate, inhibitor or prodrug composition of the present invention effective in prophylactically or therapeutically treating the disease, disorder, or other condition. In direct contact/administration formats, the composition is typically administered or transferred directly to the cells to be treated or to the tissue site of interest. In in vivo indirect contact/administration formats, the composition is typically administered or transferred indirectly to the cells to be treated or to the tissue site of interest.

Any of a variety of formats can be used to administer the compositions of the present invention (optionally along with one or more buffers and/or pharmaceutically-acceptable excipients), including inhaled administration, topical administration, transdermal administration, oral delivery, injection (e.g., by using a needle or syringe), placement within a cavity of the body (e.g., by catheter or during surgery), and the like. Pharmaceutically-acceptable excipients for use in the present invention include, but are not limited to, saline, buffered saline, dextrose, water, glycerol, ethanol, conventional nontoxic binders, disintegrants, flavorings, and carriers (e.g., pharmaceutical grades of mannitol, lactose, starch, magnesium stearate, sodium saccharine, talcum, cellulose, glucose, sucrose, magnesium, carbonate, and the like) and combinations thereof. The formulation is made to suit the mode of administration. Exemplary excipients and methods of formulation are provided, e.g., in Remington: The Science and Practice of Pharmacy, Mack Publishing Co., 20^{th} ed., 2000.

Therapeutic compositions comprising one or more flaviviral protease substrates, inhibitors or prodrugs of the invention are optionally tested in one or more appropriate in vitro and/or in vivo animal model of disease, to confirm efficacy, tissue metabolism, and to estimate dosages, according to methods well known in the art. In particular, dosages can initially be determined by activity, stability or other suitable measures of the formulation.

### EXAMPLES

The following examples are offered to illustrate, but not to limit, the scope of the present invention.

### Example 1

### Expression and Purification of Dengue NS3 Proteases

The NS3 proteases from all four dengue virus serotypes were used in the study: dengue1 (Hawaii Strain; ATCC); dengue2 (TSV01 Strain; Accession Number AY037116); dengue3 (S221/03 Strain; kind gift from Dr. Eng Eong Ooi of Environmental Health Institute, Singapore); and dengue4 (H241 Strain; ATCC). The recombinant dengue1-4 NS3 proteases (dengue CF40-gly-NS3pro1-185) were expressed in E. coli. and provided by Subhash Vasudevan and Siew Pheng Lim from the Novartis Institute of Tropical Diseases (Singapore). Briefly, the core 40 amino acid region of NS2B preceded by a molecular tag was fused in-frame with NS3 protease domain (amino acid residues 1-185) by a 9 amino acid residue (Gly4SerGly4) linker. Active recombinant dengue protease CF40-gly-NS3pro185 was expressed in BL21RIL cells. Purification was carried out via FLPC using a Hi Trap Nickel affinity column. The fractions containing the protein of interest were de-salted and further purified using gel filtration.

### Example 2

### Characterization of P1-P4 Substrate Specificity of Dengue Proteases

P4-P1 specificity of dengue NS3 serine proteases were determined using positional scanning synthetic combinatorial peptide libraries. Fluorogenic substrate libraries were synthesized as previously described in the art (Wang et al., J Biol Chem 278: 15800-8, 2003; Harris et al., Chem. Biol. 8: 1131-1141, 2001; and Harris et al., Proc Natl Acad Sci USA 97: 7754-9, 2000). Each variable position had one of 19 amino acids with cysteine excluded and methionine replaced by the isosteric amino acid norleucine (designated by small case “n” herein).

The dengue protease enzyme was diluted in assay buffer and added to the library plates. In the two-position fixed tetrapeptide substrate library, the final substrate concentration was approximately 0.25 μM/substrate/well with a total of 400 compounds per well. Accumulation of released fluorophore was monitored at 37° C. at a λex of 380 nm and a λem of 450 nm. The scanning results were shown in FIGS. 2-5, respectively, for the NS3 proteases from dengue1-4. As shown in the figures, there is a high conservation of P4-P1 substrate specificity preference among the four dengue serotypes.

### Example 3

### Determination of Prime Side Substrate Specificity of Dengue NS3 Proteases

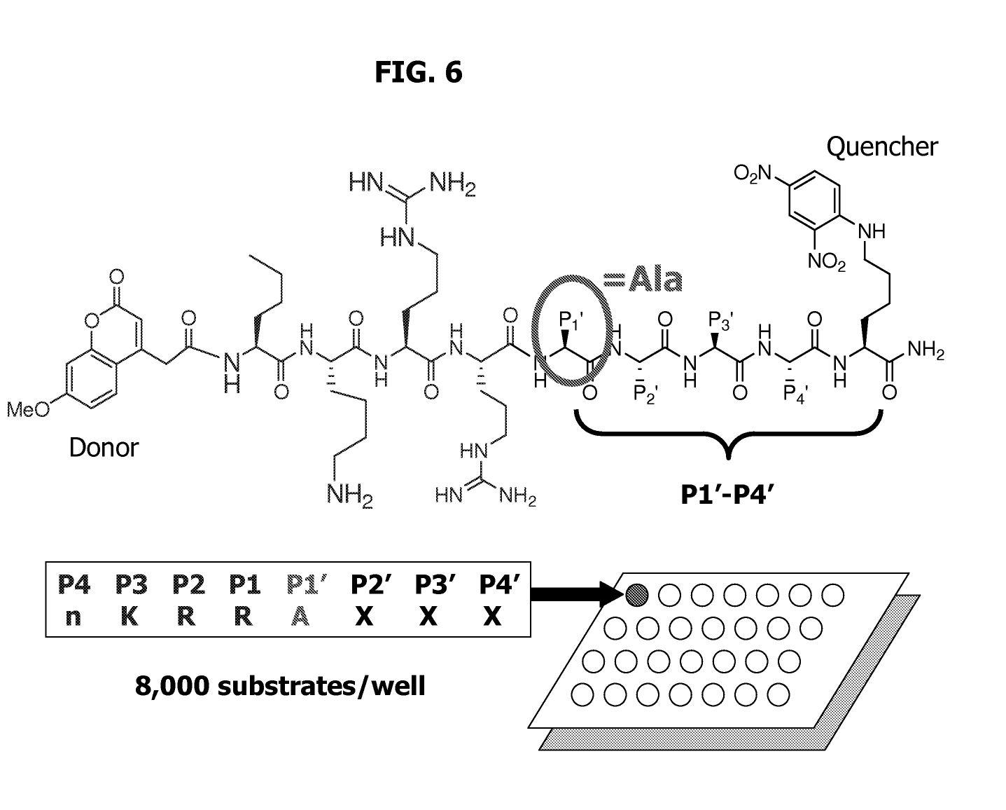

A focused octapeptide donor-quencher library was synthesized for the elucidation of the P1′-P4′ substrate specificity of dengue NS3 proteases. The non-prime sides of all the substrates in this library contained n-K-R-R (P4-P1), the optimal sequence based on the results of the P1-P4 libraries. The P1′-P4′ region of each substrate contained a tetrapeptide sequence with one position fixed and the other three varied. This library is also called P4-P1 biased, P1′-P4′ positional scanning, donor-quencher substrate library. As illustrated in FIG. 6, there are 8000 compounds in each well of the scanning libraries because each the three variable positions can be one of the 20 amino acid residues (20×20×20).

The library was synthesized in a positional scanning format using IRORI NanoKan technology (Nicolaou et al., J. Am. Chem. Soc. 122: 9953-9967, 2000). NanoKans were loaded with RINK amide AM resin that had been functionalized first with Arginine and then with Lys(DNP). The first four positions from the Lys(DNP) were varied by creating sub-libraries where each position was fixed as a particular amino acid (norleucine and all natural amino acids were included with the exception of cysteine and methionine) and the other three varied, using an isokinetic mixture (Ostresh et al., Biopolymers 34:1681-9, 1994) of 19 amino acids (norleucine and all natural amino acids were included except cysteine and methionine). This yielded 80 combinations that were easily synthesized using the ‘split and pool’ methodology, with sorting done by the IRORI sorting robot. Following the synthesis of P1′ position of the octapeptide library, the optimal non-prime sequence, nKRR, was synthesized and acylated with a fluorogenic coumarin donor group. After cleaving the substrates from a solid support, the library was lyophilized and dissolved in DMSO. For kinetic assays, 1 μl of the reconstituted library was added to 99 μl of HEPES-CHAPS buffer containing one of the dengue proteases. The final concentration of each substrate in the assay was approximately 4 nM. The increase in fluorescence intensity was measured over time at λₑₓ=320 nm and λₑₘ=380 nm with a Gemini EM plate reader (Molecular Devices). The amino acid preferences at each of the P1′-P4′ positions for the four dengue proteases are shown in FIG. 7.

### Example 4

### Dengue Protease Steady-State Kinetic Constants and Implications

This Example describes determination of the steady-state kinetic constants for the hydrolysis of substrates by the four dengue proteases. Based on the substrate specificity profile, the optimal substrate Bz-nKRR-ACMC was synthesized. Bz-nKTR-ACMC, Bz-nTRR-ACMC, Bz-TKRR-ACMC and Bz-TTRR-ACMC were also synthesized to determine the importance of the P2-P4 positions for specificity. The active site titration for each NS3 protease was performed with freshly made aprotinin (Sigma). Michaelis-Menten kinetic constants of each substrate were determined with Prizm.

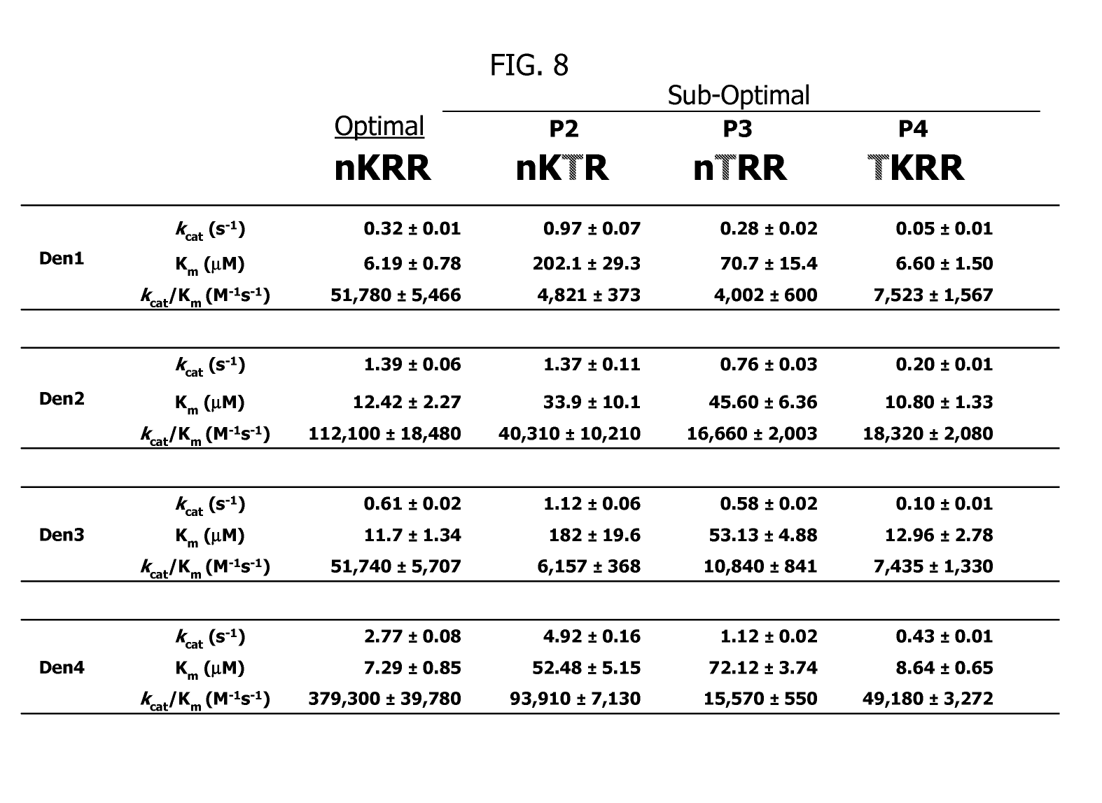

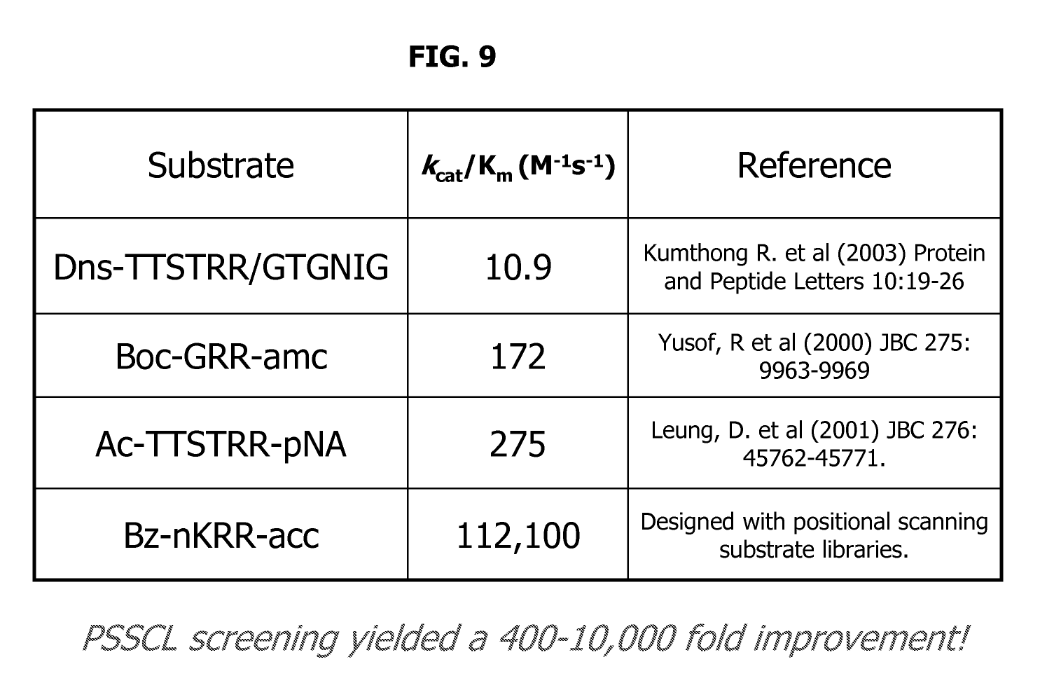

The comparative kinetic constants for substrates with the optimal and sub-optimal sequences at the P4-P1 positions are shown in FIG. 8. The results indicate that P2 and P3 contribute to ground-state binding, and that P4 contributes to transition state stabilization. The kinetics data also indicate that, compared with other substrates, substrates designed with positional scanning screening provide better tools for monitoring protease activity. As shown in FIG. 9, for dengue2 NS3 protease, such substrates yielded a 400-10,000 fold improvement over the other substrates described in the art.

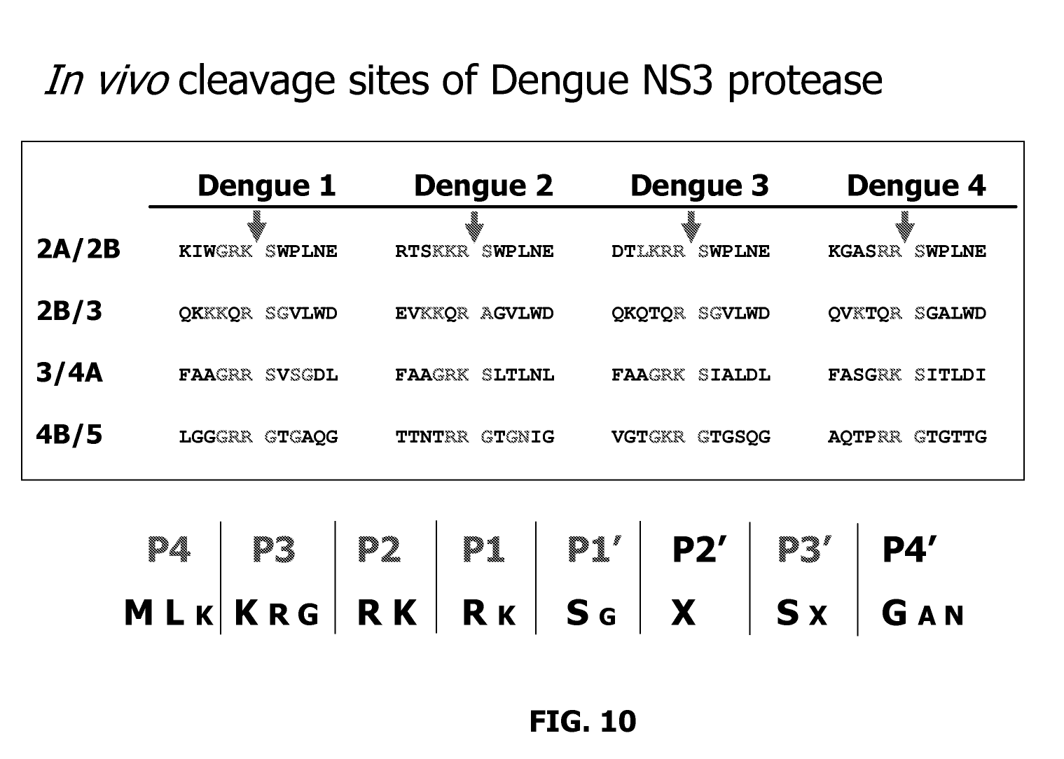

The substrate specificity information of dengue NS3 proteases also enables better design of therapeutic inhibitors. For example, the information revealed by the present inventors showed that the substrate specificity of NS3 is beyond the P2-P2′ positions and extends to P4 to P3′. The studies also showed that substrate specificities of the NS3 proteases from the four dengue serotypes are very similar. In addition, it was found that substrate specificity of NS3 proteases correlate with their physiological cleavage sites (FIG. 10).

### Example 5

### Characterization of West Nile NS3 Virus Protease

Expression and Purification of Dengue NS3 Proteases: The recombinant West Nile NS3 proteases (WN CF40-gly-NS3pro1-185) were expressed in E. coli. and provided by Subhash Vasudevan and Siew Pheng Lim from the Novartis Institute of Tropical Diseases (Singapore). Briefly, the core 40 amino acid region of NS2B preceded by a molecular tag was fused in-frame with NS3 protease domain (amino acid residues 1-185) by a 9 amino acid residue (Gly4SerGly4) linker. Active recombinant West Nile protease CF40-gly-NS3pro185 was expressed in BL21RIL cells. Purification was carried out via FLPC using a Hi Trap Nickel affinity column. The fractions containing the protein of interest were de-salted and further purified using gel filtration.

Characterization of P1-P4 Substrate Specificity of West Nile Proteases: P4-P1 specificity of West Nile NS3 serine proteases were determined using positional scanning synthetic combinatorial peptide libraries. Fluorogenic substrate libraries were synthesized as previously described in the art (Wang et al., J Biol Chem 278: 15800-8, 2003; Harris et al., Chem. Biol. 8: 1131-1141, 2001; and Harris et al., Proc Natl Acad Sci USA 97: 7754-9, 2000). Each variable position had one of 19 amino acids with cysteine excluded and methionine replaced by the isosteric amino acid norleucine (designated by small case “n” herein).

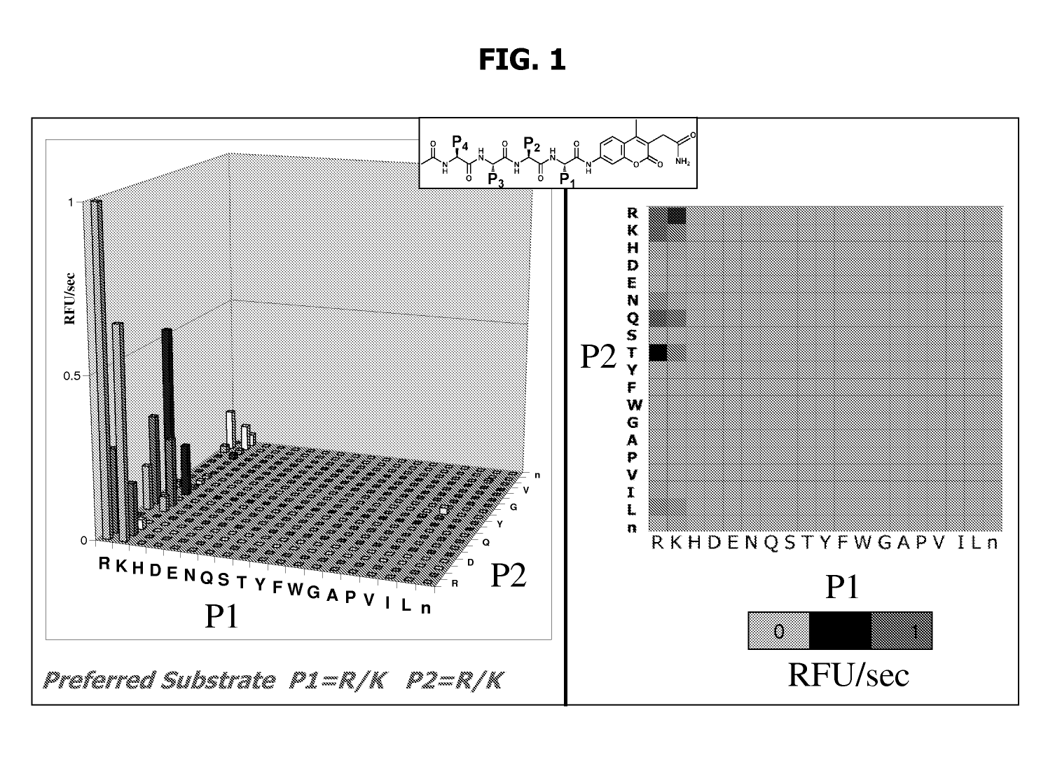

The West Nile NS3 protease enzyme was diluted in assay buffer and added to the library plates. In the two-position fixed tetrapeptide substrate library, the final substrate concentration was approximately 0.25 μM/substrate/well with a total of 400 compounds per well. Accumulation of released fluorophore was monitored at 37° C. at a λex of 380 nm and a λem of 450 nm. The scanning results were shown in FIG. 1. As shown in FIG. 11, West Nile protease shows strong preference for positively charged residues (Arg and Lys) at P1-P3 with weak selectivity at P4. The general P1-P4 subsite preferences are very similar to those observed for the four dengue serotypes (DEN1-4).

Steady-State Kinetic Constants and Implications: We further determined the steady-state kinetic constants for the hydrolysis of substrates by the West Nile NS3 proteases. Based on the substrate specificity profile, the near optimal substrate Bz-nKRR-ACMC was synthesized (Bz-nKKR-ACMC is the optimal according to the profile result). Bz-nKTR-ACMC, Bz-nTRR-ACMC, Bz-TKRR-ACMC and Bz-TTRR-ACMC were also synthesized to determine the importance of the P2-P4 positions for specificity. The active site titration for each NS3 protease was performed with freshly made aprotinin (Sigma). Michaelis-Menten kinetic constants of each substrate were determined with Prizm.

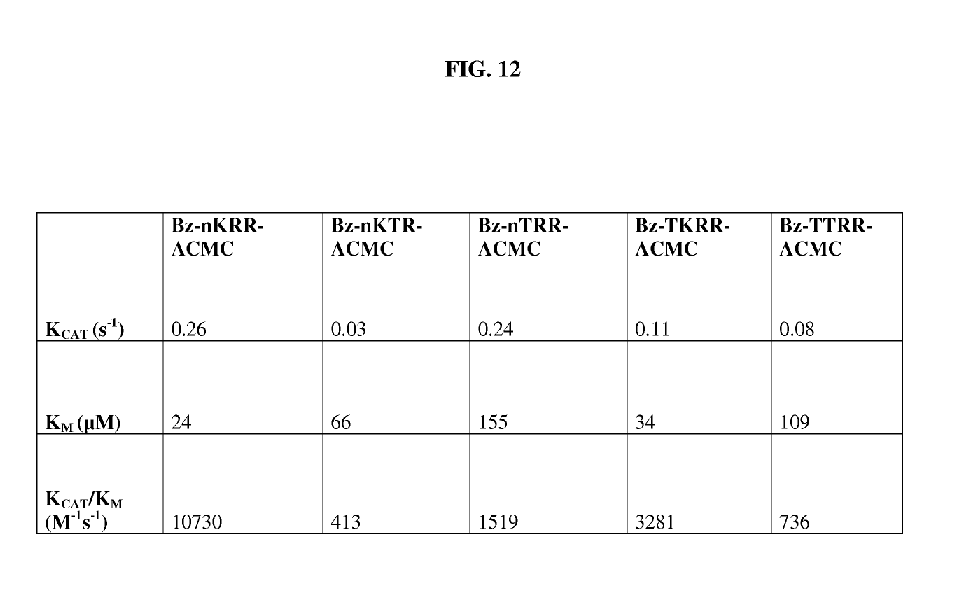

The comparative kinetic constants for substrates with the near optimal and corresponding single substitution sequences at the P4-P1 positions are shown in FIG. 12. Kinectics of West Nile protease activity on several other substrates have been reported in the literature, e.g., Nall et al., J. Biol Chem. 279:48535-42, 2004; and Chappell et al., J Biol Chem. 280:2896-903, 2005; and Shiryaev et al., Biochem J. 393:503-11, 2006. The data shown in FIG. 12 indicate that, compared with the published substrates, substrates designed with positional scanning screening provide equal or better tools for monitoring protease activity.

It is understood that the examples and embodiments described herein are for illustrative purposes only and that various modifications or changes in light thereof will be suggested to persons skilled in the art and are to be included within the spirit and purview of this application and scope of the appended claims. Although any methods and materials similar or equivalent to those described herein can be used in the practice or testing of the present invention, the preferred methods and materials are described.

All publications, patents, patent applications, and/or other documents cited in this application are incorporated by reference in their entirety and for all purposes to the same extent as if each individual publication, patent, patent application, or other document were individually so denoted.

We claim:

1. A flaviviral protease-cleavable molecule that comprises a flaviviral protease cleavage site, wherein the flaviviral protease-cleavable molecule comprises:
P₄P₃P₂P₁X
wherein:
P₁ is arginine (R) or lysine (K);
P₂ is arginine (R) or lysine (K);
P₃ is lysine (K), glycine (G), arginine (R), histidine (H), or asparagine (N);
P₄ is norleucine (n), leucine (L), lysine (K), arginine (R), or glutamine (Q); and
X comprises one or more of an inhibitory moiety, a label moiety, a polypeptide comprising 1 to 25 amino acids, or a polypeptide that is not attached to P₄P₃P₂P₁ in a naturally occurring protein; wherein P₄P₃P₂P₁ is not arginine-arginine-lysine-arginine (RRKR); and wherein the flaviviral protease cleavage site is between P₁ and X.

2. The flaviviral protease-cleavable molecule of claim 1, wherein P₁ is arginine or lysine, P₂ is arginine or lysine, P₃ is lysine, glycine, or arginine, and P₄ is norleucine, leucine, or lysine.

3. The flaviviral protease-cleavable molecule of claim 1, wherein P₄P₃P₂P₁ has a sequence selected from the group consisting of nKRR, nKKR, LKRR, LKKR, nRRR, nRKR, LRRR, LRKR, nGRR, nGKR, nKRK, nKKK, LKRK, and LKKK; and wherein “n” represents norleucine.

4. The flaviviral protease-cleavable molecule of claim 1, wherein P₄P₃P₂P₁ has a sequence of nKRR, LKRR, nKKR, or LKKR.

5. The flaviviral protease-cleavable molecule of claim 1, wherein X comprises P₁′P₂′P₃′P₄′, wherein: P₁′ is attached to P₁ and is serine or glycine; P₂′ is glycine, aspartic acid, glutamic acid, or alanine; P₃′ is serine or asparagine; and P₄′ is glycine, asparagine, or alanine.

6. The flaviviral protease-cleavable molecule of claim 5, wherein P₁′P₂′P₃′P₄′ has a sequence of SGSG, SDSG, or SESG.

7. The flaviviral protease-cleavable molecule of claim 1, wherein the label moiety comprises an absorbent, fluorescent or luminescent label moiety.

8. The flaviviral protease-cleavable molecule of claim 7, wherein the label moiety comprises a fluorophore, a coumarin moiety, or a rhodamine moiety.

9. The flaviviral protease-cleavable molecule of claim 8, wherein the coumarin moiety comprises 7-amino-4-carbamoylcoumarin, 7-amino-3-carbamoylmethyl-4-methylcoumarin, or 7-amino-4-methylcoumarin.

10. The flaviviral protease-cleavable molecule of claim 7, wherein the flaviviral protease-cleavable molecule comprises a first member of a fluorescence resonance transfer energy pair attached to the molecule on one side of the flaviviral protease cleavage site and a second member of the fluorescence resonance transfer energy pair attached to the molecule on the opposite side of the flaviviral protease cleavage site.

11. The flaviviral protease-cleavable molecule of claim 10, wherein the fluorescence resonance transfer energy pair comprises amino benzoic acid and nitro-tyrosine; 7-methoxy-3-carbamoyl-4-methylcoumarin and dinitrophenol; or 7-dimethylamino-3-carbamoyl-4-methylcoumarin and dabsyl.

12. A flaviviral protease-cleavable peptide that comprises fewer than 25 amino acids, the peptide comprising P₄P₃P₂P₁, wherein P₁ is arginine or lysine; P₂ is arginine or lysine; P₃ is lysine, glycine, arginine, histidine, or asparagine; P₄ is norleucine, leucine, lysine, arginine, or glutamine; wherein one or more amino acids is attached to either or both of P₁ and P₄; and wherein P₄P₃P₂P₁ is not arginine-arginine-lysine-arginine (RRKR).

13. The flaviviral protease-cleavable peptide of claim 12, wherein P₁ is arginine or lysine, P₂ is arginine or lysine, P₃ is lysine, glycine, or arginine, and P₄ is norleucine, leucine, or lysine.

14. The flaviviral protease-cleavable peptide of claim 12, the peptide further comprising 1 to 20 amino acids linked to P₄.

15. The flaviviral protease-cleavable peptide of claim 12, the peptide further comprising 1 to 20 amino acids linked to P₁.

16. The flaviviral protease-cleavable peptide of claim 12, the peptide further comprising P₁′P₂′P₃′P₄′, wherein P₁′ is attached to P₁ and is serine or glycine; P₂′ is glycine, aspartic acid, glutamic acid, or alanine; P₃′ is serine or asparagine; and P₄′ is glycine, asparagine, or alanine.

17. A flaviviral protease inhibitor comprising P₄P₃P₂P₁Z, wherein P₁ comprises arginine or lysine; P₂ comprises arginine, lysine, threonine, glutamine, asparagines, leucine, or isoleucine; P₃ comprises lysine, glycine, arginine, histidine, or asparagine; P₄ comprises norleucine, leucine, lysine, arginine, or glutamine; and Z comprises a transition state analog, a mechanism-based inhibitor, or an electron withdrawing group.

18. The flaviviral protease inhibitor of claim 17, wherein P₁ is arginine or lysine, P₂ is arginine or lysine, P₃ is lysine, glycine, or arginine, and P₄ is norleucine, leucine, or lysine.

19. The flaviviral protease inhibitor of claim 17, wherein P₄P₃P₂P₁ has a sequence of nKRR, LKRR, nKKR, or LKKR.

20. The flaviviral protease inhibitor of claim 17, wherein the transition state analog, mechanism-based inhibitor, or electron withdrawing moiety comprises a C-terminal aldehyde, a boronate, a phosphonate, an α-ketoamide, a chloro methyl ketone, a sulfonyl chloride, ethyl propenoate, vinyl amide, vinyl sulfone, vinyl sulfonamide.

21. A method of reducing a flaviviral protease activity in a cell, the method comprising contacting the cell with a flaviviral protease inhibitor molecule, wherein the flaviviral protease inhibitor molecule comprises P₄P₃P₂P₁Z, wherein P₁ comprises arginine or lysine; P₂ comprises arginine or lysine; P₃ comprises lysine, glycine, arginine, histidine, or asparagine; P₄ comprises norleucine, leucine, lysine, arginine, or glutamine; and Z comprises an inhibitory moiety; and wherein P₄P₃P₂P₁ is not arginine-arginine-lysine-arginine (RRKR).

22. The method of claim 21, wherein the inhibitory moiety is a transition state analog, a mechanism-based inhibitor, or an electron withdrawing group.

23. The method of claim 21, wherein the flaviviral protease is a dengue protease or West Nile protease.

24. The method of claim 21, wherein the cell is in a mammal.

25. The method of claim 21, wherein the cell is in a human subject.

26. The method of claim 21, wherein the flaviviral protease inhibitor is applied to the cell in a pharmaceutically acceptable excipient.
